(19) United States
(12) Patent Application Publication
(10) Pub. No.: US 2021/0361730 A1
(43) Pub. Date: Nov. 25, 2021

(54) PREPARATIONS CONTAINING ANTHOCYANINS FOR USE IN THE INFLUENCE OF CARDIOVASCULAR CONDITIONS

(71) Applicant: Evonik Operations GmbH, Essen (DE)

(72) Inventors: Rosario LIZIO, Dieburg (DE); Sara LIEBANA VIÑAS, Aschaffenburg (DE); Abdelhak ZOUBIR, Darmstadt (DE); Michael MUMA, Darmstadt (DE); Tim SCHAECK, Sindelfingen (DE)

(73) Assignee: Evonik Operations GmbH, Essen (DE)

(21) Appl. No.: 17/049,446

(22) PCT Filed: Apr. 18, 2019
(86) PCT No.: PCT/EP2019/060136

§ 371 Date: Oct. 21, 2020

(30) Foreign Application Priority Data
Apr. 23, 2018 (EP) 18168770.8

(51) Int. Cl. A61K 36/45; A61K 36/185; A61K 31/7048; A61P 9/12

(52) U.S. Cl. CPC A61K 36/45; A61K 36/185; A61K 2236/51; A61P 9/12; A61K 2236/55; A61K 31/7048

(57) ABSTRACT

The present invention is related to preparations containing one or more anthocyanins for use in the prevention and treatment of cardiovascular diseases and reduction of arterial stiffness in a subject.

The present invention is related to preparations containing one or more anthocyanins for use in the prevention and treatment of cardiovascular diseases and reduction of arterial stiffness in a subject.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes, Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, and apples and vegetables such as red cabbage. Bilberries, in particular Vaccinium myrtillus, and black currants, in particular Ribes nigrum, are especially suitable.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and—to a lesser extent—malvidin, peonidin, and petunidin (cyanidin-3-O-glucoside (C3G), delphinidin-3-O-glucoside (D3G), malvidin-3-O-glucoside (M3G), peonidin-3-O-glucoside and petunidin-3-O-glucoside). Flavonols include quercetin- and kaempferol-glucosides. The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but delphinidin- and cyanidin-3-O-glucoside are also found (Gafner, Bilberry—Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

The present invention is related to a preparation for use in the prevention and treatment of cardiovascular diseases and reduction of arterial stiffness in a subject, wherein the preparation comprises an extract of black currants and bilberries.

With increasing age, the blood vessels usually become stiffer compared to those of a young person. This phenomenon occurs primarily because elastin in blood vessels' walls deteriorates and is replaced by collagen, which is less flexible. The increased stiffness causes the blood to travel faster through the vessels, therefore arterial stiffness is strongly correlated to the pulse wave velocity PWV. If a person's arterial stiffness is higher than the normal value for their age, this is a determinant of hypertension, i.e. increased systolic and diastolic blood pressure. Since hypertension is an increasingly large problem, arterial stiffness is of interest as well. As increased arterial stiffness can be detected before hypertension occurs, this allows to start treatment or behavioral changes early, possibly avoiding hypertension. It is also well known that atherosclerotic plaques and aneurysms involve changes in vessel wall properties and therefore their stiffness (M. McGarry et al., “In vivo repeatability of the pulse wave inverse problem in human carotid arteries”, J. of biomechanics, vol. 64, pp. 136-144, 2017). Also in this case, an accurate arterial stiffness measurement, in particular its variation, improves diagnosis and monitoring of the connected diseases. Various cardiovascular parameters can be analyzed to gain information about a person's cardiovascular health.

According to the present invention one or more cardiovascular conditions are improved selected from blood pressure (BP), pulse wave velocity (PWV), vascular age index (AgIx), augmentation index (AIx) and heart rate variability (HRV), preferably the augmentation index (AIx).

Blood pressure (BP, measured in mmHg) denotes the pressure that the blood traveling through a large artery exerts onto its walls. Blood pressure is given in two numbers, the systolic blood pressure (maximal pressure during one cardiac cycle) and the diastolic blood pressure (minimal pressure during one cardiac cycle). Hypertension is a major risk factor for multiple diseases, such as stroke and end-stage renal disease, and overall mortality. By the year 2025, it is expected that the number of people across the world who are hypertensive will have risen to 1.56 billion. If the condition is detected early and treated properly, the risk of disease can be decreased significantly. Therefore, it is important to measure BP regularly in order to detect abnormal changes. Besides this, a change of lifestyle can often decrease BP and prevent hypertension, provided that a tendency towards it is detected early. Currently, there exist several different approaches to measure BP. The most common device is an inflatable cuff that is placed at the patient's arm and that applies pressure onto the brachial artery. While this allows an accurate measurement, it is perceived as inconvenient by some patients and it requires a visit to a doctor or the purchase of a device. Other approaches are invasive, such as intravenous cannula that are placed inside an artery. Those are only used in a clinical context, e.g. during a surgery.

Pulse wave velocity (PWV, measured in m/s) describes the velocity of blood that travels through a person's arteries and is used as a measure of arterial stiffness. It is estimated by the time difference of two sensors at a known distance. The most precise devices to measure PWV perform a carotid-femoral measurement. For this measurement, one tonometer is placed at the carotid artery which is located at the neck and a second tonometer is placed at the femoral artery at the upper leg. Those tonometers measure the pressure pulse waves of the arteries. From the time difference between the signals and the distance between the tonometers, PWV can be calculated. PWV can also be measured with only one blood pressure cuff at the brachial artery. The PWV is then estimated from the delay between the first peak (forward wave) and the reflected wave.

Vascular age index (AgIx) is a cardiovascular parameter that gives information on the age condition of the arteries. The index describes the cardiovascular age of a person. It should be lower than the person's chronological age if their vessels aged slower than average and higher than their chronological age otherwise. It can be determined with devices that use an inflatable cuff. Augmentation index (AIx) is a cardiovascular parameter that is usually obtained from a pressure pulse wave and can be measured at a large artery with a device that uses an inflatable cuff. Just like arterial stiffness, the augmentation index increases with age and can be used to estimate the risk of suffering from a cardiovascular disease in the future.

Heart rate variability (HRV) describes the variation in the time interval between heartbeats and is usually calculated from an ECG. Heart rate variability is an indicator for the body's ability to recover and response to stress and indirectly related to arterial stiffness.

In a preferred embodiment, the augmentation index (AIx) is reduced in the subject after a period of 30 days, wherein when calculating a linear regression model, the slope of the linear has a negative value, preferably not more than −0.05 more preferably not more than −0.08.

In a preferred embodiment, the black currants are the fruit of Ribes nigrum and/or the bilberries are the fruit of Vaccinium myrtillus. It is further preferred, when the composition contains an extract from black currants and bilberries in a weight ratio of 0.5:1 to 1:0.5. In an advantageous configuration of the present invention, the composition is an extract of the pomaces from black currants and bilberries.

It is particularly preferred, when the composition comprises anthocyanins and the anthocyanins are present in the composition at a concentration of at least 25 weight-%, preferably at least 30 weight-%, or at least 35 weight-%, or at least 40 weight-%, or at least 45 weight-%, or at least 50 weight-%.

It is preferred, according to the present invention, when the extract is an alcoholic extract, preferably a methanol extract. The extract is preferably produced by a process comprising the steps of
extraction of black currants and/or bilberries,
purification via chromatography,
mixing of the extract(s) with water and
spray-drying of the mixture.

One example of such a process is disclosed in EP1443948.

In a preferred embodiment, maltodextrin is added to the composition.

The preparation according to the present invention preferably contains at least three monosaccharide anthocyanins. Moreover, it preferably contains at least one monosaccharide anthocyanin in which the saccharide is arabinose or at least one disaccharide anthocyanin in which the disaccharide is rutinose. The composition preferably contains anthocyanins with at least two different aglycones, more preferably at least four. Especially preferably the composition contains anthocyanins in which the aglycone units are cyanidin, peonidin, delphinidin, petunidin, malvidin and optionally also pelargonidin. In one preferred embodiment, the composition also contains at least one trisaccharide anthocyanin. The disaccharide anthocyanins are more water-soluble than the monosaccharides; moreover cyanidin and delphinidin anthocyanins are amongst the most water-soluble anthocyanins.

In an advantageous embodiment of the present invention anthocyanins are selected from cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, delphinidin-3-rutinoside. The anthocyanins are preferably selected from cyanidin-3-glucoside, cyanidin-3-rutinoside, delphinidin-3-glucoside, delphinidin-3-rutinoside, cyanidin-3-galactoside, delphinidin-3-galactoside.

It is especially preferred according to the present invention to use a preparation comprising one or more of the following anthocyanins: cyanidin-3-glucoside, delphinidin-3-glucoside, cyanidin-3-rutinoside and delphinidin-3-rutinoside. In a further preferred configuration, the preparation comprising cyanidin-3-glucoside, delphinidin-3-glucoside, cyanidin-3-rutinoside and delphinidin-3-rutinoside.

The anthocyanins can be from natural sources or from synthetic productions. Natural sources are preferably selected from fruits, flowers, leaves, stems and roots, preferably violet petal, seed coat of black soybean. Preferably anthocyanins are extracted from fruits selected from: açai, black currant, aronia, eggplant, blood orange, marion blackberry, black raspberry, raspberry, wild blueberry, cherry, queen Garnet plum, red currant, purple corn (Z. mays L.), concord grape, norton grape, muscadine grape, red cabbage, okinawan sweet potato, Ube, black rice, red onion, black carrot. Particularly suitable fruit sources for the anthocyanins are cherries, bilberries, blueberries, black currants, red currants, grapes, cranberries, strawberries, black chokeberry, and apples and vegetables such as red cabbage. Bilberries, in particular Vaccinium myrtillus, and black currants, in particular Ribes nigrum, are especially suitable. It is further preferred to use plants enriched with one or more of anthocyanins as natural sources, preferably plants enriched with delphinidin-3-rutinoside.

The counterion in the anthocyanins in the product of the invention may be any physiologically tolerable counteranions, e.g. chloride, succinate, fumarate, malate, maleate, citrate, ascorbate, aspartate, glutamate, etc. Preferably however the counterion is a fruit acid anion, in particular citrate, as this results in the products having a particularly pleasant taste.

Besides the anthocyanins, the products of the process of the invention may desirably contain further beneficial or inactive ingredients, e.g. vitamins (in particular vitamin C), flavones, isoflavones, anticoagulants (e.g. maltodextrin, silica, etc.), desiccants, etc. Desirably however the anthocyanins constitute at least 50% by weight of the product compositions, excluding the coating material.

In an advantageous configuration of the present invention the cardiovascular conditions influenced are selected from blood pressure (BP), pulse wave velocity (PWV), vascular age index (AgIx), augmentation index (AIx) and heart rate variability (HRV), preferably the augmentation index (AIx).

It is preferred when the composition comprises anthocyanins and is to be administered to the subject in a dose of the anthocyanins/regimen of 1 to 10 oral dosages of at least 80 mg anthocyanins each per day, preferably 3 to 6 oral dosages of at least 80 mg anthocyanins each per day.

Moreover, a preparation containing a liquid extract from anthocyanins from Vaccinium myrtillus and Ribes nigrum in a weight ratio of 0.5:1 to 1:0.5 for improving the cardiovascular health status of an animal or a human being, preferably the augmentation index (AIx), is part of the present invention.

### Experiment: Effect of Anthocyanins on Cardiovascular Parameters

The anthocyanin rich extract (Medox®; Biolink Group AS, Sandnes, Norway) used in the present study is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus).

The total anthocyanin content was 80 mg/capsule, which consisted of 17 different natural purified anthocyanins from bilberry (Vaccinium myrtillus) and blackcurrant (Ribes nigrum). The relative content of each anthocyanin was as follows: 33.0% of 3-O-b-rutinoside, 3-O-b-glucosides, 3-O-b-galactosides, and 3-O-b-arabinosides of cyanidin; 58.0% of 3-O-b-rutinoside, 3-O-b-glucosides, 3-O-b-galactosides, and 3-O-b-arabinosides of delphinidin; 2.5% of 3-O-b-glucosides, 3-O-b-galactosides, and 3-O-b-arabinosides of petunidin; 2.5% of 3-O-b-glucosides, 3-O-b-galactosides, and 3-O-b-arabinosides of peonidin; 3.0% of 3-O-b-glucosides, 3-O-b-galactosides, and 3-O-b-arabinosides of malvidin. The 3-O-b-glucosides of cyanidin and delphinidin constituted at least 40-50% of the total anthocyanins.

The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside.

In addition to the anthocyanins mentioned above, the product also contained maltodextrin (around 40 weight-% of the composition), and citric acid (to maintain stability of anthocyanins). The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

The dose of anthocyanins was determined based on previous animal studies and a recent human studies. Supplementation of 130 mg anthocyanins per kg and per day contained in black rice anthocyanin extract was shown to significantly improve the lipid profile in mice (Xia X, Ling W, Ma J, et al. An anthocyanin-rich extract from black rice enhances atherosclerotic plaque stabilization in apolipoprotein E-deficient mice. J Nutr 2006; 136:2220-5). The effective dose of anthocyanins was determined to be in the range of 100.5˜335.0 mg/d for a 70-kg human being when it was extrapolated to the humans by the method of specific surface area. In the human study (Karlsen A, Retterstol L, Laake P, et al. Anthocyanins inhibit nuclear factor-kappaB activation in monocytes and reduce plasma concentrations of pro-inflammatory mediators in healthy adults. J Nutr 2007; 137:1951-4), daily intake of 320 mg anthocyanins was found to be safe and effective in improving the inflammatory response.

### Experimental Setup

A study using the anthocyanin rich extract Medox® was conducted which spanned over 30 days and comprised between three and seven measurements within this timespan that were taken from 27 subjects (under 35 years old).

The measurements were conducted using the clinical device “Mobil-O-Graph PWA” (I.E.M. GmbH). This device works similar to a standard measurement device for blood pressure, applying a cuff to the subject's upper arm. The inflatable cuff exerts pressure onto the upper arm's brachial artery and measures not only the blood pressure, but also performs a pressure pulse wave analysis (PWA).

It obtains the pressure pulse wave from at the upper arm (brachial artery) and also estimates the aortal pressure wave. The vascular parameters that were obtained are both systolic and diastolic blood pressure, pulse wave velocity, vascular age index and augmentation index.

### Analysis of Effect of Anthocyanin Rich Extract Medox®

The effect of the anthocyanin rich extract Medox® on the study's subjects using one-sample and two-sample t-tests with 95% significance level. The criterion to be evaluated in the t-tests is the slope of a linear regression model that has been calculated for each cardiovascular parameter over all individual measurements. The parameters of the linear regression models, including the slopes of the linear, were calculated for each subject and each parameter over the course of measurements using the method of least squares.

First, the mean of slope for each cardiovascular parameter was analysed and it was tested whether this slope in the group has changed from zero or not. A change in the slope of the linear indicates a change regarding this parameter over the time measured. A negative slope indicates an improvement of cardiovascular health. Then, it was tested whether there was a significant difference within the group over the course of measurements of 30 days.

TABLE 1
Mean of the slope for each cardiovascular parameter

| Parameter | |
|---|---|
| Systolic blood pressure | −0.09 |
| Diastolic blood pressure | 0.03 |
| Pulse rate variability | 0 |
| Vascular age index | −0.04 |
| Mean heart rate | −0.02 |
| Augmentation index | −0.08 |

The mean of slopes for the different cardiovascular parameters are listed in table 1. For most values a negative slope was detected. These data show that small decreases of the systolic blood pressure, vascular age index, mean heart rate and augmentation were measured, which indicates an improvement in cardiovascular health. This shows that several parameters of cardiovascular health are improved after Medox® intake.

In a second step, the h and p values of the one-sample t-test were analysed, whether the mean of their slopes significantly differ from the normal distribution, i.e. a constant slope implies no trend over the duration of the study. The h and p values for the different cardiovascular parameters are summarized in table 2. An h-value of 1 shows that significance level of the one-sample t-test was exceeded.

TABLE 2
One-sample t-test result for each cardiovascular parameter

| Parameter | h-value | p-value |
|---|---|---|
| Systolic blood pressure | 0 | 0.39 |
| Diastolic blood pressure | 0 | 0.45 |
| Pulse rate variability | 0 | 0.21 |
| Vascular age index | 0 | 0.23 |
| Mean heart rate | 0 | 0.72 |
| Augmentation index | 1 | 0.05 |

For the group of subjects analysed, a significant effect of Medox® for the augmentation index could be proven by a one-sample t-test with 95% significance and a group size of 27. The linear equations over time and the slope of the equations for the 27 subjects are summarized in table 3.

TABLE 3
Linear equation over time t in days and slope of equation for
Augmentation Index (AIx) measured in 27 subjects over 30 days

| Subject | Linear equation over time t | Slope of the equation |
|---|---|---|
| 1 | f(t) = 0.1791*t + 15.3080 | 0.1791 |
| 2 | f(t) = −0.1370*t + 15.8356 | −0.1370 |
| 3 | f(t) = 0.0529*t + 9.8740 | 0.0529 |
| 4 | f(t) = −0.1422*t + 12.2664 | −0.1422 |
| 5 | f(t) = 0.0230*t + 26.3099 | 0.0230 |
| 6 | f(t) = −0.0086*t + 20.7657 | −0.0086 |
| 7 | f(t) = −0.3238*t + 26.3695 | −0.3238 |
| 8 | f(t) = 0.1195*t + 10.3543 | 0.1195 |
| 9 | f(t) = 0.0605*t + 20.5493 | 0.0605 |
| 10 | f(t) = −0.3113*t + 21.9695 | −0.3113 |
| 11 | f(t) = −0.1548*t + 16.9455 | −0.1548 |
| 12 | f(t) = −0.1852*t + 17.5296 | −0.1852 |
| 13 | f(t) = 0.1051*t + 20.1235 | 0.1051 |
| 14 | f(t) = 0.1077*t + 5.7153 | 0.1077 |
| 15 | f(t) = −0.0204*t + 8.8900 | −0.0204 |
| 16 | f(t) = −0.0351*t + 18.7795 | −0.0351 |
| 17 | f(t) = 0.1979*t + 7.4512 | 0.1979 |
| 18 | f(t) = −0.1165*t + 13.8321 | −0.1165 |
| 19 | f(t) = −0.0863*t + 31.9447 | −0.0863 |
| 20 | f(t) = −0.5974*t + 19.6466 | −0.5974 |
| 21 | f(t) = 0.0899*t + 18.6585 | 0.0899 |
| 22 | f(t) = −0.5491*t + 23.1049 | −0.5491 |
| 23 | f(t) = −0.1549*t + 20.0921 | −0.1549 |
| 24 | f(t) = −0.1235*t + 13.0530 | −0.1235 |
| 25 | f(t) = 0.2103*t + 15.5430 | 0.2103 |
| 26 | f(t) = −0.2640*t + 26.4468 | −0.2640 |
| 27 | f(t) = −0.1785*t + 13.1073 | −0.1785 |

| | |
|---|---|
| Average slope (27 subjects) | −0.0831 |

1. A method for preventing and treating a cardiovascular disease, improving a cardiovascular condition and reducing-arterial stiffness in a subject, the method comprising:
administering a preparation comprising an extract of black currants and bilberries to the subject in need thereof.

2. The method according to claim 1, wherein the cardiovascular condition is at least one selected from the group consisting of blood pressure, pulse wave velocity, vascular age index, augmentation index and heart rate variability

3. The method according to claim 2, wherein the augmentation index is reduced in the subject after a period of 30 days, and
wherein when calculating a linear regression model, a slope of a linear has a negative value.

4. The method according to claim 1, wherein the preparation further comprises an anthocyanin and the anthocyanin is present in the preparation at a concentration of at least 25 weight-%.

5. The method according to claim 4, wherein the anthocyanin is at least one selected from the group consisting of cyanidin-3-glucoside, cyanidin-3-galactoside, cyanidin-3-arabinoside, delphinidin-3-glucoside, delphinidin-3-galactoside, delphinidin-3-arabinoside, petunidin-3-glucoside, petunidin-3-galactoside, petunidin-3-arabinose, peonidin-3-glucoside, peonidin-3-galactoside, peonidin-3-arabinose, malvidin-3-glucoside, malvidin-3-galactoside, malvidin-3-arabinose, cyanidin-3-rutinoside, and delphinidin-3-rutinoside.

6. The method according to claim 1, wherein the preparation further comprises cyanidin-3-glucoside, delphinidin-3-glucoside, cyanidin-3-rutinoside and delphinidin-3-rutinoside.

7. The method according to claim 1, wherein the black currants are a fruit of Ribes nigrum and/or the bilberries are a fruit of Vaccinium myrtillus.

8. The method according to claim 1, wherein the preparation comprises an extract of pomaces from black currants and bilberries.

9. The method according to claim 8, wherein the extract is prepared by a process comprising extracting black currants and/or bilberries, purifying via chromatography, mixing the extract(s) with water to form a mixture and spray-drying the mixture.

10. The method according to claim 1,
wherein the preparation further comprises an anthocyanin and
the preparation is administered to the subject 1 to 10 oral dosages of at least 80 mg anthocyanins each per day.

11. A preparation comprising a liquid extract from an anthocyanin from Vaccinium myrtillus and Ribes nigrum in a weight ratio of 0.5:1 to 1:0.5.
